(19) United States
(12) Patent Application Publication
(10) Pub. No.: US 2021/0019835 A1
(43) Pub. Date: Jan. 21, 2021

(54) EFFICIENT TREATMENT SELECTION USER INTERFACE

(71) Applicants: David Williams, Minnetonka, MN (US); Brenda Reinhardt, Mahtomedi, MN (US); Cheryl Warner, Eden Prairie, MN (US); Gretchen Poffenberger, Stillwater, MN (US); Scott Heintzeman, Greenfield, MN (US)

(72) Inventors: David Williams, Minnetonka, MN (US); Brenda Reinhardt, Mahtomedi, MN (US); Cheryl Warner, Eden Prairie, MN (US); Gretchen Poffenberger, Stillwater, MN (US); Scott Heintzeman, Greenfield, MN (US)

(21) Appl. No.: 16/925,515

(22) Filed: Jul. 10, 2020

Related U.S. Application Data
(60) Provisional application No. 62/874,206, filed on Jul. 15, 2019.

(51) Int. Cl. G06Q 40/08; G16H 10/65; G06Q 10/10; G16H 70/20; G06F 16/9535; G06F 16/9538; G06F 3/0482

(52) U.S. Cl. CPC G06Q 40/08; G16H 10/65; G06Q 10/10; G06F 3/0482; G06F 16/9535; G06F 16/9538; G16H 70/20

(57) ABSTRACT

A method includes downloading searchable data to a mobile device, the searchable data comprising coverage statements for treatments wherein each coverage statement is associated with a combination of state, company, plan, diagnosis and treatment. A user interface is displayed on the mobile device providing controls each configured to receive a respective selection of a state, company, plan, diagnosis and treatment. The selections of the state, company, plan, diagnosis and treatment are used to search the searchable data and to retrieve at least one coverage statement. The at least one coverage statement is displayed on the user interface.

### CROSS-REFERENCE OF RELATED APPLICATION

The present application is based on and claims the benefit of U.S. provisional patent application Ser. No. 62/874,206, filed Jul. 15, 2019, the content of which is hereby incorporated by reference in its entirety.

### BACKGROUND

The amount of time a physician has to spend with a patient during an office visit is quite limited. During that time, the physician must evaluate the patient's current status, determine a diagnosis if a diagnosis has not been previously determined, and develop a course of treatment that best suits the patient.

One complicating factor is that each patient has a separate insurance plan and different insurance plans cover different available treatments. In fact, for a given state, there are thousands of available insurance plans. Given the limited time available, the physician is unable to wade through the documentation for the patient's insurance plan to determine which treatments the patient's plan covers.

The discussion above is merely provided for general background information and is not intended to be used as an aid in determining the scope of the claimed subject matter. The claimed subject matter is not limited to implementations that solve any or all disadvantages noted in the background.

### SUMMARY

A method includes downloading searchable data to a mobile device, the searchable data comprising coverage statements for treatments wherein each coverage statement is associated with a combination of state, company, plan, diagnosis and treatment. A user interface is displayed on the mobile device providing controls each configured to receive a respective selection of a state, company, plan, diagnosis and treatment. The selections of the state, company, plan, diagnosis and treatment are used to search the searchable data and to retrieve at least one coverage statement. The at least one coverage statement is displayed on the user interface.

In accordance with a further embodiment, a mobile device includes a display, a memory, and a processor executing instructions to perform steps. The steps include displaying a user interface on the display with the user interface providing controls configured to receive selections of a state, a company from a list of companies designated for the state, a selection of a plan from a list of plans designated for the combination of the state and company, a diagnosis, and a treatment. Upon a selection of the state, company, plan, diagnosis and treatment, a search is automatically performed to retrieve and display at least one coverage statement without requiring further selection of a control to request the at least one coverage statement.

In accordance with a still further embodiment, a method includes displaying a user interface capable of receiving selections of a state, a company, a plan, a diagnosis and a treatment and upon determining that a state, a company, a plan, a diagnosis and a treatment have been selected, automatically displaying a coverage statement describing whether the treatment is covered by the selected plan issued by the selected company in the selected state for the selected diagnosis without requiring a further input.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1 depicts a user interface in accordance with one embodiment.

FIG. 2 is a flow diagram of a method of using the user interface of FIG. 1.

FIG. 3 is a flow diagram of a method of updating and deploying data to mobile devices in accordance with one embodiment.

FIG. 4 is a block diagram of a system that performs the steps of FIG. 3.

FIG. 5 is a block diagram of a mobile device that can be used with various embodiments.

FIG. 6 is a block diagram of a computing device that can be used as a server in the various embodiments.

### DETAILED DESCRIPTION

Embodiments herein provide an efficient user interface that allows a physician to quickly determine which of a collection of treatments are covered by a patient's health insurance. In particular, the user interface allows a physician to quickly select a state, company, plan, diagnosis and treatment and in response receive a coverage statement that indicates whether the treatment is covered by the selected plan for the selected company within the selected state without having to select a separate control to request the coverage statement. Thus, as soon as the application has received all of the information it needs to determine if coverage is available, it automatically performs a search to obtain coverage statements that indicate whether coverage is available. This improves the technology of the user interface by making the user interface easier and faster to use.

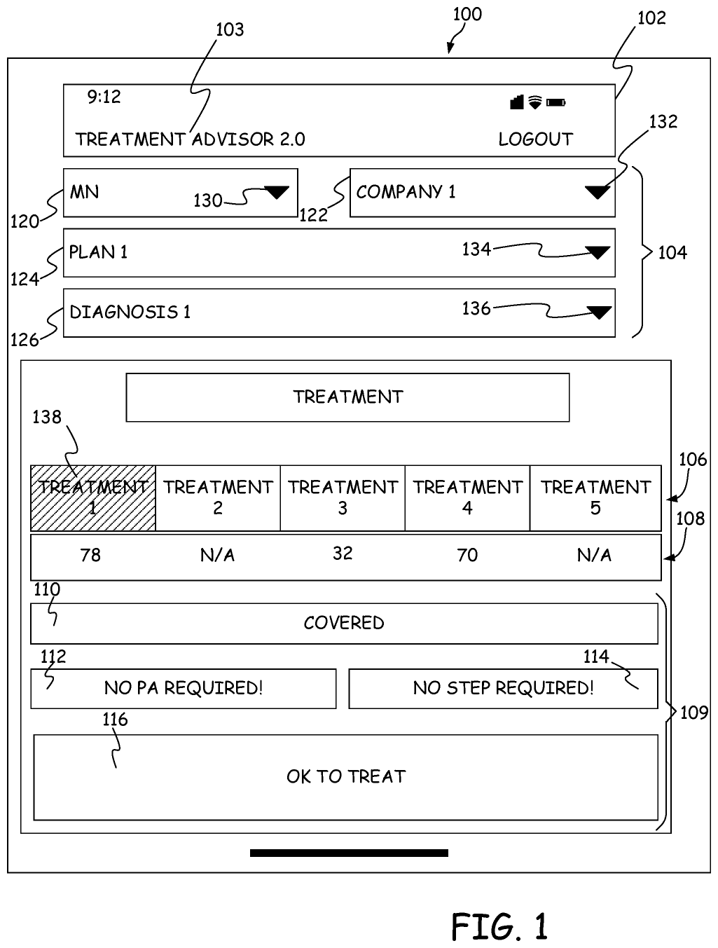

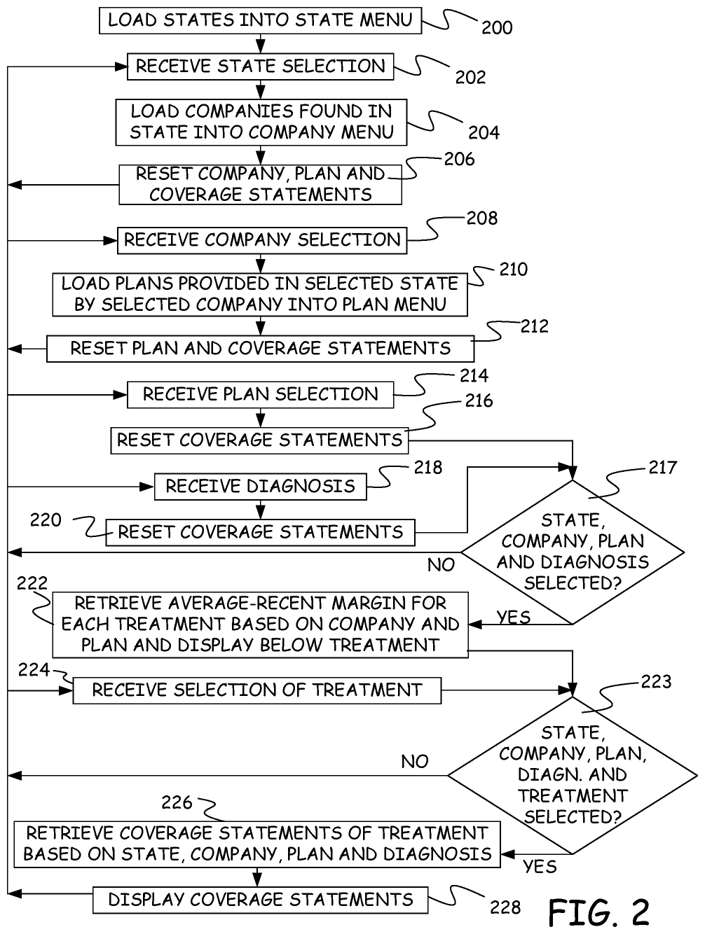

FIG. 1 shows a user interface 100 that is displayed on a display of a mobile device and that is used to obtain coverage statements in accordance with one embodiment. FIG. 2 provides a flow diagram of a method of using the user interface of FIG. 1 to obtain coverage statements. User interface 100 includes a header 102, a coverage criteria section 104 a treatment selection area 106, an optional margin area 108, and a coverage statement area 109.

Header 102 includes an application title 103. Coverage criteria area 104 includes a state selection box 120, a company selection box 122, a plan selection box 124 and a diagnosis selection box 126. Coverage statement area 109 is composed of a covered/not covered designation 110, a prior-authorization designation 112, a steps-required designation 114 and a notes area 116.

The method of FIG. 2 begins when the application corresponding to user interface 100 is selected on a mobile device. Upon activation of the application, a current list of states that the application is equipped to handle is loaded into a menu control associated with state selection box 120 at step 200. Upon selection of pulldown control 130 of state selection box 120, the menu control displays the list of states such that the user can select one of the states.

At step 202, one of the states listed in the state menu control is selected by the user and this new state selection is received by the application and is placed in state selection box 120. At step 204, a list of all companies found in the selected state is loaded into a menu control associated with company selection box 122 such that when a pulldown control 132 is selected, the menu control displays the list of companies so that the user can select one of the states. In accordance with one embodiment, the list of companies for a state are retrieved from a file that specifically lists the companies for the state. In other embodiments, the list of companies is determined by performing a search of a database containing records that include a state field and a company field to identify all records that contain the state in the state field. The list of companies is then built from the companies that appear in the company field of those records.

At step 206, any company and plan that have been previously selected in company selection box 122 and plan selection box 124 are removed due to the selection of the new state since the previously selected company and plan may no longer be valid for the newly selected state. In addition, all of the designations in coverage statement area 109 are reset so that no values are shown in the designation areas, such as covered/not covered designation 110, prior-authorization designation 112, steps-required designation 114 and notes area 116.

At step 208, the application receives the selection of a company listed in the menu control associated with company selection box 122 and the selected company is then displayed in company selection box 122. At step 210, the application loads a list of plans provided by the selected company into a menu control associated with plan selection box 124. When a pulldown control 134 is selected, the menu control displays the list of plans so that the user of the application can select one of the plans. In accordance with one embodiment, the list of plans for a company are retrieved from a file that specifically lists the plans for the company. In other embodiments, the list of plans is determined by performing a search of a database containing records that include a state field, a company field and a plan field to identify all records that contain the selected state in the state field and the selected company in the company field. The list of plans is then built from the plans that appear in the plan field of those records.

At step 212, any previously selected plan in plan selection box 124 is reset and all of the designations (if any) in coverage statement area 109 are reset so that no values are shown in the designation areas, such as covered/not covered designation 110, prior-authorization designation 112, steps-required designation 114 and notes area 116.

At step 214, the application receives a selection of a plan through the menu control associated with plan selection box 124 and at step 216 the application resets the designations (if any) in coverage statement area 109 so that no values are shown in the designation areas, such as covered/not covered designation 110, prior-authorization designation 112, steps-required designation 114 and notes area 116.

After step 216, the application determines if a state, company, plan and diagnosis have been selected at step 217. If one of these criteria has not been selected yet, the application returns to wait for the selection of the diagnosis or the selection of a different state, company or plan.

At step 218, the application receives the selection of a diagnosis through a menu control associated with diagnosis box 126. In accordance with one embodiment, when a pulldown control 136 is selected, the menu control associated with diagnosis box 126 displays a list of diagnoses so that the user can select one of the diagnoses. At step 220, the application resets the designations (if any) in coverage statement area 109 so that no values are shown in the designation areas, such as covered/not covered designation 110, prior-authorization designation 112, steps-required designation 114 and notes area 116.

After step 220, the application returns to step 217 to determine if a state, company, plan and diagnosis are currently selected. If all of these criteria are currently selected at step 217, the application continues at optional step 222 where a margin for each treatment in drug selection area 106 is retrieved based on the state, company, plan, and diagnosis selected in user interface 100. As noted in the discussion below, this margin is determined based on recent payments received for the drug under the selected company and plan for the selected diagnosis. The margins are then displayed beneath the corresponding treatments in margin area 108. If no payment has been received for a treatment, the margin is set to N/A.

After step 222, the application determines if a state, company, plan, diagnosis and treatment are currently selected at step 223. If one or more of these criteria have not been selected, the application returns to wait for a treatment to be selected or for a new state, company, plan or diagnosis to be selected.

At step 224, a selection of one of the treatments in treatment selection area 106 is received. When a treatment is selected, the treatment is highlighted as shown by highlighted treatment 138 of FIG. 1. In accordance with one embodiment, the list of treatments in treatment selection area is the same for all combinations of state, company, plan and diagnosis. In accordance with one embodiments, treatments in treatment selection area 106 include one or more of oral medications, injectable medications, topical medications, stimulation therapies, physical therapies, and surgeries. After step 224, the application proceeds to step 223 to determine if a state, company, plan, diagnosis and treatment are currently selected.

When all of these criteria have been set, the application searches searchable data to locate and retrieve coverage statement(s) for the selected treatment based on the selected state, company, plan, and diagnosis at step 226. In accordance with one embodiment, the search is automatically performed any time a state, company, plan, diagnosis and treatment have been selected without requiring the user to select an additional control to request the coverage statements. Thus, the embodiments improve the efficiency of the user interface by not requiring the user to take an additional action beyond setting the state, company, plan, diagnosis and treatment in order to receive the coverage statement(s) for that combination of criteria.

In accordance with one embodiment, the searchable data comprises a database that has been downloaded onto the same mobile device executing the application. In other embodiments, the searchable data comprises one or more data files. In still further embodiments, the searchable data comprises a data structure loaded into Random Access Memory. The coverage statement(s) are then displayed in coverage statement area 109 at step 228 without further input from the user.

In accordance with one embodiment, the retrieved coverage statements include at a minimum whether payment for the treatment is covered by the selected plan offered by the selected company in the selected state for the selected diagnosis. The coverage statements may also include an indication that preauthorization is required and/or steps that must be taken before prescribing the treatment.

For example, within coverage statement area 109 of FIG. 1, covered/not covered designation 110 indicates whether the selected plan issued by the selected company in the selected state covers payment for the selected treatment for the selected diagnosis. In accordance with one embodiment, when the selected plan covers payment (either partial payment or full payment), covered/not covered designation 110 contains the phrase “covered” and when the selected plan does not cover payment, designation 110 contains the phrase “not covered”. If designation 110 contains the phrase “not covered” the remaining designations 112, 114 and 116 in coverage statement area 109 are blank.

When a treatment is covered, prior-authorization designation 112 indicates whether prior authorization is required before prescribing the treatment. If prior authorization is required, area 112 includes the phrase “PA Required!” and if prior authorization is not required, area 112 includes the phrase “No PA Required!”

When a treatment is covered, steps-required designation 114 indicates whether any steps must be performed before prescribing the treatment. If no steps are required, steps-required designation 114 includes the phrase “No Step Required!”. When steps are required, steps-required designation 114 includes the phrase “Step Required!” and notes area 116 includes a description of the steps that must be performed.

After step 228, the application returns to wait for a selection of a new state, company, plan, diagnosis or treatment. If a new plan is selected at step 214 without changing any of the other selections, a new search is automatically performed at step 226 to retrieve the coverage statements for the new plan and the new coverage statements are displayed at step 228. Note that the new search is performed without requiring the user to select an additional control to request the coverage statements. Instead, simply selecting a new plan is enough to trigger the search and display of new coverage statements.

In FIG. 2, step 208 and step 214 are depicted as being accessible at any time. In accordance with one embodiment, however, a company can only be selected at step 208 when a state is currently selected and a plan can only be selected at step 214 when both a state and a company are currently selected.

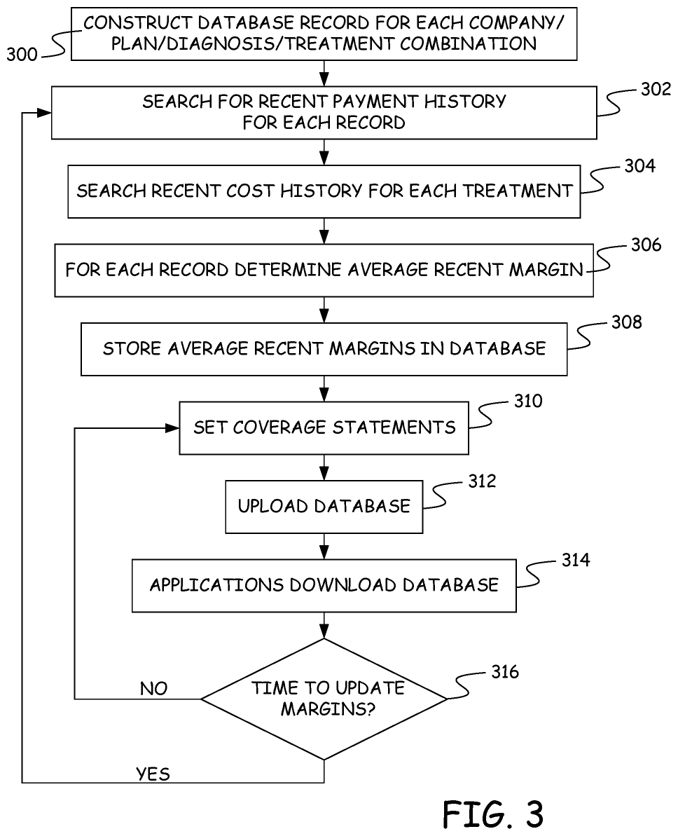

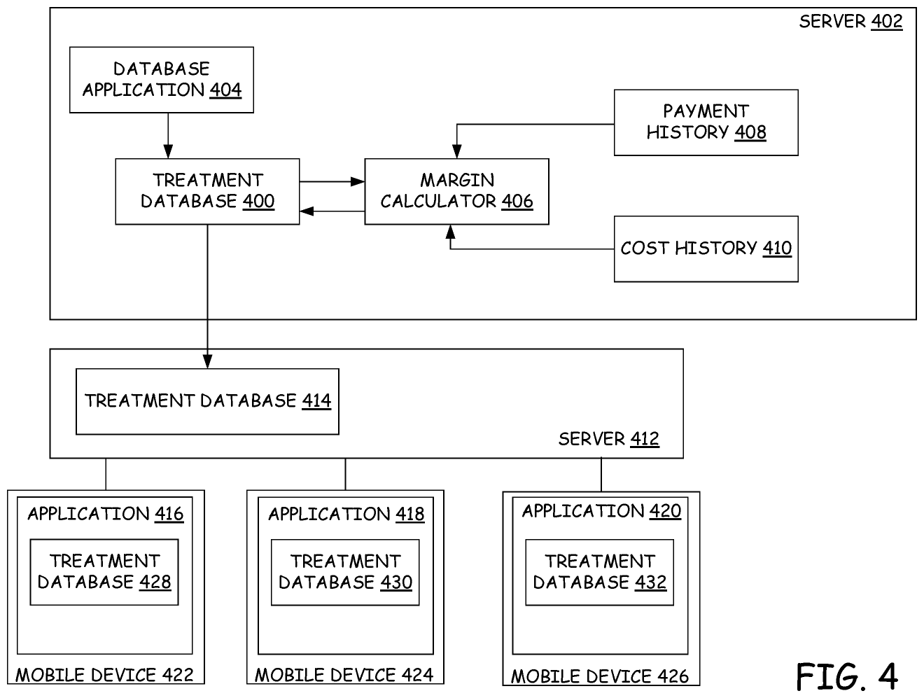

FIG. 3 provides a flow diagram of a method of constructing and deploying the database used in the method of FIG. 2. FIG. 4 provides a block diagram of elements used to construct and deploy the database.

In step 300 of FIG. 3, a database record is constructed in a treatment database 400 on a server 402 using a database application 404. In step 300, a separate record is created in the database for each combination of state, company, plan, diagnosis and treatment.

At step 302, a margin calculator 406 searches for recent payments that are associated with each record in a payment history 408. To perform this search, margin calculator 406 first selects a record in treatment database 400. Margin calculator 406 then searches payment history 408 for all payments that have the same combination of state, company, plan, diagnosis and treatment as the record selected from treatment database 400. In accordance with one embodiment, a payment for a combination of state, company, plan, diagnosis and treatment is the total amount received in response to sending an invoice for the treatment and may include the sum of amounts sent by different entities including private insurance companies, government agencies and private individuals. In accordance with one embodiment, the payment history is limited to payments made in the current calendar year.

At step 304, margin calculator 406 searches a cost history 410 to determine a recent cost for each treatment. At step 306, margin calculator 406 determines an average recent margin for each combination of state, company, plan, diagnosis and treatment by taking the difference between each recent payment for that combination and the recent cost to form a margin associated with each payment and then averaging those margins across all payments for that combination. At step 308, margin calculator 406 stores the average recent margins in treatment database 400.

At step 310, database application 404 is used to set coverage statements for each combination of state, company, plan, diagnosis and treatment. These coverage statements include whether the treatment is covered, whether prior authorization is required, whether steps are required and if steps are required, a description of the steps. At step 312, treatment database 400 is uploaded to a distribution server 412 to produce an accessible treatment database 414.

At step 314, applications, such as applications 416, 418 and 420 on mobile devices, such as mobile devices 422, 424 and 426, download accessible treatment database 414 to produce local treatment databases 428, 430 and 432. Applications 416, 418 and 420 then use the respective local treatment database 428, 430 and 432 to perform the method of FIG. 2 and to generate the user interface of FIG. 1.

At step 316, margin calculator 406 determines whether it is time to update the margins for each entry. If it is not time to update the margins, the process returns to step 310 on a regular basis, such as daily, to set coverage statements for one or more of the combinations of state, company, plan, diagnosis and treatment since at least one such combination may have its coverage parameters changed in that period of time. When it is time to update the margins at step 316, the process returns to step 302 and a new search for recent payments is performed and steps 304-308 are then repeated before the coverage statements are updated at step 310.

Although treatment databases are discussed in the embodiment of FIGS. 3 and 4, in other embodiments, the searchable data is stored as a collection of files or messages that are made available to the mobile devices.

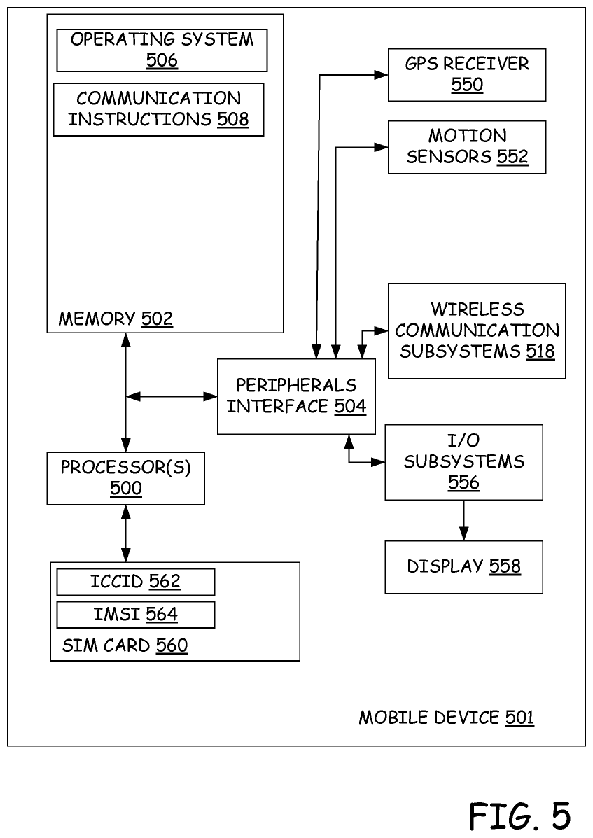

FIG. 5 illustrates a block diagram of mobile device 501 that can be used to implement the various embodiments. Mobile device 501 includes one or more processors 500, such as a central processing unit or image processors, and a memory 502. Processor(s) 500 and memory 502 are connected by one or more signal lines or buses. Memory 502 can take the form of any processor-readable medium including a disk or solid-state memory, for example. Memory 502 includes an operating system 506 that includes instructions for handling basic system services and performing hardware-dependent tasks. In some implementations, operating system 506 can be a kernel. Memory 502 also includes various instructions representing applications that can be executed by processor(s) 500 including communication instructions 508 that allow processor 500 to communicate through peripherals interface 504 and wireless communication subsystems 518 to a wireless cellular telephony network and/or a wireless packet switched network.

Peripherals interface 504 also provides access between processor(s) 500 and one or more of a GPS receiver 550, motion sensors 552, and input/output subsystems 556. GPS receiver 550 receives signals from Global Positioning Satellites and converts the signals into longitudinal and latitude information describing the location of mobile device 501. The position of mobile device 501 may also be determined using other positioning systems such as Wi-Fi access points, television signals and cellular grids. Motion sensors 552 can take the form of one or more accelerometers, a magnetic compass, a gravity sensor and/or a gyroscope. Motion sensors 552 provide signals indicative of movement or orientation of mobile device 501. I/O subsystems 556 control input and output for mobile device 501. I/O subsystems 556 can include a touchscreen display 558, which can detect contact and movement or break thereof using any of a plurality of touch sensitivity technologies including, but not limited to capacitive, resistive, infrared and surface acoustic wave technologies as well as other proximity sensor arrays or other elements for determining one or more points of contact with display 558. Other inputs can also be provided such as one or more buttons, rocker switches, thumb wheel, infrared port, USB port and/or pointer device such as a stylus.

Mobile device 501 can also include a subscriber identity module, which in many embodiments takes the form of a SIM card 560. SIM card 560 stores an ICCID 562 and an IMSI 564. ICCID 562 is the Integrated Circuit Card Identifier, which uniquely identifies this card on all networks. IMSI 564 is the international mobile subscriber identity, which identifies the SIM card on an individual cellular network. When communicating through wireless communication subsystems 518, processor(s) 500 can use identifiers 562 and/or 564 to uniquely identify mobile device 501 during communications. In accordance with many embodiments, SIM card 560 is removable from mobile device 501 and may be inserted in other devices.

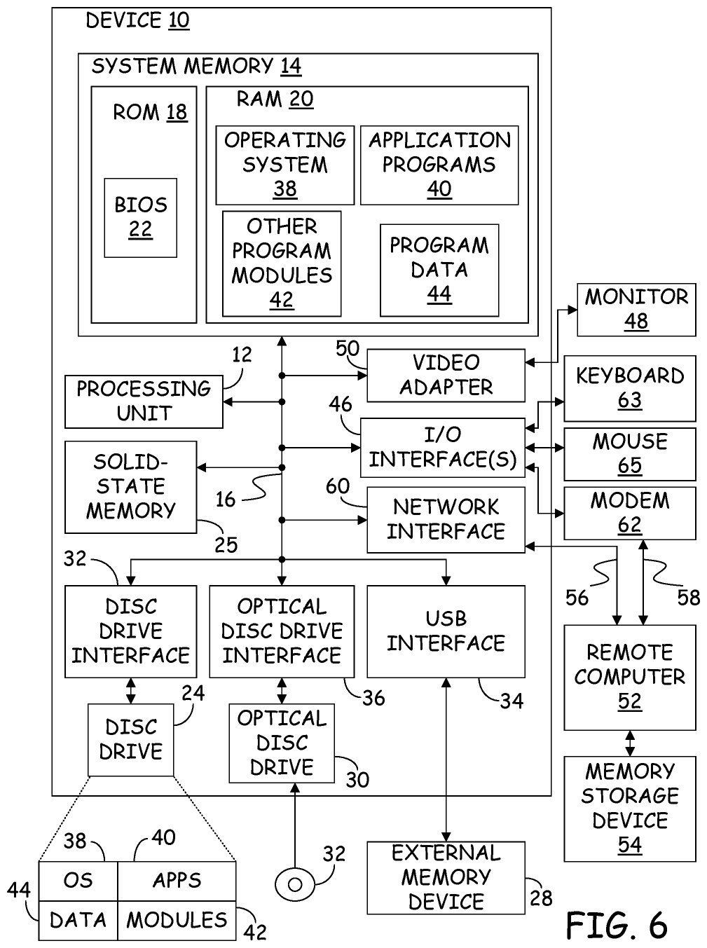

FIG. 6 provides an example of a computing device 10 that can be used to implement one or more of the servers discussed above. Computing device 10 includes a processing unit 12, a system memory 14 and a system bus 16 that couples the system memory 14 to the processing unit 12. System memory 14 includes read only memory (ROM) 18 and random-access memory (RAM) 20. A basic input/output system 22 (BIOS), containing the basic routines that help to transfer information between elements within the computing device 10, is stored in ROM 18. Computer-executable instructions that are to be executed by processing unit 12 may be stored in random access memory 20 before being executed.

Computing device 10 further includes an optional hard disc drive 24, an optional external memory device 28, and an optional optical disc drive 30. External memory device 28 can include an external disc drive or solid-state memory that may be attached to computing device 10 through an interface such as Universal Serial Bus interface 34, which is connected to system bus 16. Optical disc drive 30 can illustratively be utilized for reading data from (or writing data to) optical media, such as a CD-ROM disc 32. Hard disc drive 24 and optical disc drive 30 are connected to the system bus 16 by a hard disc drive interface 32 and an optical disc drive interface 36, respectively. The drives and external memory devices and their associated computer-readable media provide nonvolatile storage media for the computing device 10 on which computer-executable instructions and computer-readable data structures may be stored. Other types of media that are readable by a computer may also be used in the exemplary operation environment.

A number of program modules may be stored in the drives and RAM 20, including an operating system 38, one or more application programs 40, other program modules 42 and program data 44. In particular, application programs 40 can include programs for implementing any one of the applications discussed above. Program data 44 may include any data used by the systems and methods discussed above.

Processing unit 12, also referred to as a processor, executes programs in system memory 14 and solid-state memory 25 to perform the methods described above.

Input devices including a keyboard 63 and a mouse 65 are optionally connected to system bus 16 through an Input/Output interface 46 that is coupled to system bus 16. Monitor or display 48 is connected to the system bus 16 through a video adapter 50 and provides graphical images to users. Other peripheral output devices (e.g., speakers or printers) could also be included but have not been illustrated. In accordance with some embodiments, monitor 48 comprises a touch screen that both displays input and provides locations on the screen where the user is contacting the screen.

The computing device 10 may operate in a network environment utilizing connections to one or more remote computers, such as a remote computer 52. The remote computer 52 may be a server, a router, a peer device, or other common network node. Remote computer 52 may include many or all of the features and elements described in relation to computing device 10, although only a memory storage device 54 has been illustrated in FIG. 6. The network connections depicted in FIG. 6 include a local area network (LAN) 56 and a wide area network (WAN) 58. Such network environments are commonplace in the art.

The computing device 10 is connected to the LAN 56 through a network interface 60. The computing device 10 is also connected to WAN 58 and includes a modem 62 for establishing communications over the WAN 58. The modem 62, which may be internal or external, is connected to the system bus 16 via the I/O interface 46.

In a networked environment, program modules depicted relative to the computing device 10, or portions thereof, may be stored in the remote memory storage device 54. For example, application programs may be stored utilizing memory storage device 54. In addition, data associated with an application program may illustratively be stored within memory storage device 54. It will be appreciated that the network connections shown in FIG. 6 are exemplary and other means for establishing a communications link between the computers, such as a wireless interface communications link, may be used.

Although elements have been shown or described as separate embodiments above, portions of each embodiment may be combined with all or part of other embodiments described above.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms for implementing the claims.

What is claimed is:

1. A method comprising:
downloading searchable data to a mobile device, the searchable data comprising coverage statements for treatments wherein each coverage statement is associated with a combination of state, company, plan, diagnosis and treatment;
displaying a user interface on the mobile device providing controls each configured to receive a respective selection of a state, company, plan, diagnosis and treatment;
using the selection of the state, company, plan, diagnosis and treatment to search the searchable data and to retrieve at least one coverage statement; and
displaying the at least one coverage statement on the user interface.

2. The method of claim 1 wherein the control configured to receive a selection of a treatment comprises a list of a plurality of selectable treatments.

3. The method of claim 2 wherein the list of the plurality of selectable treatments is displayed while displaying the at least one coverage statement for a treatment currently selected in the displayed list.

4. The method of claim 3 wherein the at least one coverage statement comprises a statement that the treatment is covered by the selected plan issued by the selected company for the selected state.

5. The method of claim 4 wherein the at least one coverage statement further comprises a statement that prior authorization is required for coverage.

6. The method of claim 4 wherein the at least one coverage statement further comprises a statement that steps must be performed before prescribing the treatment in order for the treatment to be covered.

7. The method of claim 1 further comprising receiving a selection of a different company and in response removing all coverage statements from the user interface.

8. The method of claim 7 further comprising:
receiving a selection of a different plan after receiving the selection of the different company and in response:
using the selection of the state, the different company, the different plan, the diagnosis and the treatment to search the searchable data and to retrieve at least one second coverage statement; and
displaying the at least one second coverage statement.

9. A mobile device comprising:
a display;
a memory; and
a processor executing instructions to perform steps comprising:
displaying a user interface on the display, the user interface providing:
a control configured to receive a selection of a state;
a control configured to receive a selection of a company from a list of companies designated for the state;
a control configured to receive a selection of a plan from a list of plans designated for the combination of the state and company;
a control configured to receive a diagnosis; and
a control to receive a selection of a treatment;
wherein upon a selection of the state, company, plan, diagnosis and treatment, a search is automatically performed to retrieve and display at least one coverage statement without requiring further selection of a control to request the at least one coverage statement.

10. The mobile device of claim 9 wherein upon receiving selection of a second company the at least one coverage statement is removed from the display.

11. The mobile device of claim 10 wherein upon receiving selection of a second plan after receiving selection of the second company, a second search is automatically performed to retrieve and display a second coverage statement associated with the second company and second plan without requiring further selection of a control to request the second coverage statement.

12. The mobile device of claim 9 wherein the at least one coverage statement indicates coverage for the treatment.

13. The mobile device of claim 9 wherein the at least one coverage statement indicates that prior approval is required for coverage.

14. The mobile device of claim 9 wherein the at least one coverage statement indicates that particular steps must be performed before treatment in order for the treatment to be covered.

15. The mobile device of claim 9 wherein the search comprises a search of data stored in the memory that is updated periodically.

16. A method comprising:
displaying a user interface capable of receiving selections of a state, a company, a plan, a diagnosis and a treatment; and
upon determining that a state, a company, a plan, a diagnosis and a treatment have been selected, automatically displaying a coverage statement describing whether the treatment is covered by the selected plan issued by the selected company in the selected state for the selected diagnosis without requiring a further input.

17. The method of claim 16 wherein the selection of the company is limited by the selection of the state.

18. The method of claim 17 wherein the selection of the plan is limited by the selection of the state and the selection of the company.

19. The method of claim 16 wherein displaying a user interface capable of receiving selections of a treatment comprises displaying a same list of selectable treatments for every combination of selected state, company and plan.

20. The method of claim 19 further comprising displaying an average margin for a treatment based on a selected state, company and plan.
